(19) United States
(12) Patent Application Publication
(10) Pub. No.: US 2008/0039847 A1
(43) Pub. Date: Feb. 14, 2008

(54) Implant and system for stabilization of the spine

(76) Inventors: Mark Piper, West Chester, PA (US); Benjamin Jackson, Chadds Ford, PA (US)

Correspondence Address: CARTER, DELUCA, FARRELL & SCHMIDT, LLP, 445 BROAD HOLLOW ROAD, SUITE 225, MELVILLE, NY, 11747, US

(21) Appl. No.: 11/890,830

(22) Filed: Aug. 8, 2007

Related U.S. Application Data
(60) Provisional application No. 60/821,848, filed on Aug. 9, 2006.

(51) Int. Cl. A61B 17/58; A61B 17/56

(52) U.S. Cl. 606/69; 606/72

(57) ABSTRACT

According to an aspect of the present disclosure, a spinal plating system for facilitating stabilization of the spine is provided. The spinal plating system comprises superior and inferior plate members for positioning against exterior surfaces of respective superior and inferior vertebrae and in contacting relation therewith. The superior and inferior plate members include cooperating contacting surfaces adapted to engage each other to maintain a minimum predetermined distraction distance between the superior and inferior vertebrae and to permit at least one of relative articulating movement, pivoting movement or lateral movement of the superior and inferior plate members. The spinal plating system further includes at least one fastener for mounting each of the superior and inferior plate members to the respective superior and inferior vertebrae.

### CROSS-REFERENCE TO RELATED APPLICATION

The present application claims the benefit of and priority to U.S. Provisional application Ser. No. 60/821,848, filed on Aug. 9, 2006, the entire content of which is incorporated herein by reference.

### BACKGROUND

1. Technical Field

The present disclosure relates to surgical implants and/or plating systems and, more particularly, to surgical implants and/or plating systems used for the stabilization of the spine of an individual.

2. Background of Related Art

Pain in the spine may be caused by a number of different factors, including and not limited to degeneration of the disc and/or stenosis. Degeneration of the disc can lead to tears in the annulus and/or a reduction in a height of a disc. The tears in the annulus can be a source of pain eventually requiring treatment or they can be asymptomatic. The reduction in the height of the disc can lead to tightening of the neural foramen of the spinal canal, which can pinch or otherwise irritate the nerve roots causing pain which may eventually require treatment.

With each of these conditions one solution is for surgeons to fuse vertebra of the spine, the current standard of care for pain persisting after 3 to 6 months of conservative care. To fuse the spine the surgeon immobilizes the segment to be fused with instrumentation. The instrumentation can be pedicle screws, plates, metallic spacers and/or allograft spacers. Bone grafting material is often used as well. Most often the surgeon will attempt to restore some of the disc height, thereby opening the lateral foramen of the spinal canal and reducing the irritation of the nerve roots.

There are drawbacks to fusion. The most significant is that fusion of the vertebra eliminates motion at the fused segments, causing the adjacent levels of vertebra to compensate for the loss of motion by becoming hyper-mobile. It is theorized that this increases the rate of degeneration of the hyper-mobile discs.

Because of the drawbacks of fusion, various alternative techniques and devices have been developed to avoid fusion if at all possible. One of the first attempts was the artificial disc. These devices are implanted anteriorly and fit in the space between the two vertebra in place of the disc. To implant either device the surgeon must remove a portion of or the entire disc, distract the space between the two remaining vertebra and implant the artificial disc into the space. These devices are effective in that they do distract the two vertebra and maintain motion. The distraction relieves the posterior impingement and the motion preservation theoretically prevents adjacent level disease. However the devices are not effective for all spine ailments. The devices typically do not provide resistance to motion and are not effective for treatment of spinal instability, nor do they allow for compression as does a natural disc. Additionally, the anterior approach in the lumber spine requires significant dissection to reach the spine, which can lead to further complications. Finally, most of the disc must first be removed to place the artificial disc, and this plus the potential for scarring from the difficult surgery limits revision options for the surgeon if the replacement fails.

These short-comings of the artificial disc have caused others to seek better alternatives. Many are lumbar devices with a posterior approach. The posterior approach is generally considered more favorable in the lumbar spine because surgeons are familiar with the approach and the relatively short dissection required. One of these systems is the Graf ligament. The Graf ligament is a tension band made of braided polymer applied between two screws placed in pedicles at adjacent levels, one ligament on each side. The ligaments limit flexion but have no effect on extension. They also tend to force the spine to translate as the patient tries to flex. This narrows the diameter of the neural foramen through which the nerve roots pass and increases load transfer to the posterior part of the disc. It is known that increased load transfer by the posterior annulus can produce annular tear.

A second device is the Dynesys device detailed in European Patent No. EP 0669109. It utilizes a braided polyester band affixed to two pedicle screws to prevent excessive flexion. The disc is distracted by insertion of a polymer tube around the polyester braid, preventing collapse. It also prevents excessive lordosis, but it applies no distraction to the anterior portion of the disc. Its distraction of the posterior, but not the anterior of the disc space causes a reduction of lordosis or even kyphosis. It is well known that a kyphotic segment in the lumbar spine can produce back pain.

In U.S. Pat. No. 5,415,661 to Holmes, systems and devices intended to treat spinal instability are disclosed. This device is an implant used to control or dampen motion. It does not require distraction and therefore does not cause excessive kyphosis, but, if not distracted, disc height is not increased and pain from posterior stenosis would not be relieved. A surgeon would not be precluded from distracting posteriorly, but with the disadvantage of inducing kyphosis or a reduction in lordosis.

The anterior approach is generally considered best for the cervical spine as the dissection is relatively easy, with little incision required and a simple retraction of the trachea to expose the anterior of the cervical spine. This makes placing an artificial disc significantly easier in the cervical spine than in the lumbar. Because the anterior approach is more common in cervical surgeries there has been less criticism of the artificial cervical disc, but, many of the artificial disc disadvantages are not solved by the anterior approach. For example, placing of the disc in the cervical spine still requires removal of much of the disc, limiting revision options. The discs also force the cervical spine to move according to the bearing surface of the implant, not as intended by the facets and soft tissue, which can lead to degeneration in the facets in the cervical spine. In fact some problems are amplified in the cervical spine, such as, implant dislocation. This problem was first solved through the addition of a central keel. This, however, has raised concerns that it could split the vertebral body during or after implantation. This is even a greater concern with disc implantation at multiple adjacent levels.

As mentioned, the anterior approach to cervical fusion is the most common. The device most used for immobilizing the cervical spine is a cervical plate and/or a cage. Plates have been used for many years to immobilize bone segments to promote fracture healing or arthrodesis. Plates that allow some motion have also been used in the cervical spine. These plates all limit motion but do allow some settling of the construct between implantation and when the levels instrumented fuse. There are various theories behind allowing the settling motion from stressing the bone to allow formation (Wolfe's Law) to simply forcing direct graft to bone contact using the patient's weight to do so. All of the plates were invented and or developed for stabilizing the bone segments while fusion occurs. Cervical plates using super elastic materials to limit bone motion which may be used for fusion or non-fusion are known, however, their invention is limited to use of super elastic materials. A major concern is that devices designed to move through bending of materials eventually lead to failure of the material.

Accordingly, a need exists for surgical implants which overcome at least some of the afore-mentioned deficiencies.

### SUMMARY

The present disclosure relates to surgical implants and/or plating systems used for the stabilization of the spine of an individual.

According to an aspect of the present disclosure, a spinal plating system for facilitating stabilization of the spine is provided. The spinal plating system includes superior and inferior plate members for positioning against exterior surfaces of respective superior and inferior vertebrae and in contacting relation therewith. The superior and inferior plate members include cooperating contacting surfaces adapted to engage each other to maintain a minimum predetermined distraction distance between the superior and inferior vertebrae and to permit at least one of relative articulating movement, pivoting movement or lateral movement of the superior and inferior plate members. The spinal plating system further includes at least one fastener for mounting each of the superior and inferior plate members to the respective superior and inferior vertebrae.

The superior and inferior plate members may include corresponding stop surfaces to limit the at least one of relative articulating movement, pivoting movement or lateral movement of the superior and inferior plate members.

In an embodiment, one of the superior and inferior plate members may include a stop depending therefrom and the other of the superior and inferior plate members may include inner surface portions defining a recess for receiving the stop. In use, the stop is engageable with the inner surface portions to limit the at least one of relative articulating movement, pivoting movement or lateral movement of the superior and inferior plate members.

The contacting surface may be generally arcuate.

In use, the positioning against exterior surfaces of respective superior and inferior vertebrae may be accomplished at an anterior surface, a posterior surface, a lateral anterior surface or a lateral posterior surface of said vertebrae.

Each of the superior and inferior plate members may be formed in a plurality of pieces which are configured for selective mating with one another.

At least one of the superior plate member and the inferior plate member may include a pin projecting from a surface thereof and configured for operative engagement with a complementary feature formed on the other of the superior plate member and the inferior plate member. The complementary feature may be a window having a dimension, and wherein the movement of the pin within the window may be defined by the dimensions of the window.

In an embodiment, one of the superior and the inferior plate member may include a hollow formed in a surface thereof, and the other of the superior and the inferior plate member may include an enlarged head portion configured for receipt within the hollow. The hollow may include a rim formed at least partially around an end thereof for inhibiting removal of the head portion from within the hollow. The head portion may be separable from the respective superior and inferior plate member.

The spinal plating system may further include an inter-engagement member operatively connecting the superior plate member and the inferior plate member to one another. The inter-engagement member may be rotatably connected to the superior plate member, and may be at least one of rotatably and slidably connected to the inferior plate member. In an embodiment, the inferior plate member may define a cut-out and may include a bar extending across the cut-out, wherein the inter-engagement member may be rotatably and slidably supported on the bar of the inferior plate member.

In an embodiment, one of the superior and the inferior plate members includes at least one recess formed in a side edge thereof, and the other of the superior and inferior plate members includes at least one respective tongue portion configured and dimensioned for slidable engagement in a respective recess. It is contemplated that a pair of spaced apart recesses may be provided, and that a pair of complementary spaced apart tongues may be provided. The recesses and the tongues may have a substantially arcuate longitudinal axis. The tongues may be substantially parallel to one another.

It is contemplated that at least one of the superior and the inferior plate member may include at least one plug projecting from a surface thereof for engagement with a vertebra.

According to another aspect of the present disclosure, a spinal plating system for facilitating stabilization of adjacent vertebrae is provided and includes superior and inferior plate members for mounting against respective superior and inferior vertebrae of a spine in contacting relation therewith. The superior and inferior plate members include cooperating contacting surfaces adapted to engage each other to maintain a minimum predetermined distraction distance between the superior and inferior vertebrae and to permit relative movement of the superior and inferior plate members along at least two of an x-axis, y-axis and z-axis of the superior and inferior plate members. The spinal plating system further includes at least one fastener for mounting each of the superior and inferior plate members to the respective superior and inferior vertebrae.

According to yet another embodiment of the present disclosure, a spinal plating system for facilitating stabilization of adjacent vertebrae is provided and includes superior and inferior plate members for mounting against respective superior and inferior vertebrae of a spine in contacting relation therewith. The superior and inferior plate members are joined to one another by an intermediate portion for maintaining a minimum predetermined distraction distance between the superior and inferior vertebrae and for permitting relative movement of the superior and inferior plate members along at least one of an x-axis, y-axis and z-axis of the superior and inferior plate members. The spinal plating system further includes at least one fastener for mounting each of the superior and inferior plate members to the respective superior and inferior vertebrae.

The intermediate portion may be formed of a resilient material. At least one of the superior and inferior plate member may include a stop member configured to prevent movement of the superior plate member and the inferior plate member relative to one another after the superior plate member and the inferior plate member have move a predetermined distance relative to one another.

The intermediate portion may include at least one of at least one side cut formed in side surfaces thereof and at least one passage extending therethrough.

The intermediate portion may be formed in one of the superior plate member and the inferior plate member and may be made of nitinol or other elastic or flexible material including at least one passage formed therein.

### DETAILED DESCRIPTION OF THE DRAWINGS

The present invention will become more fully understood from the description and the accompanying drawings, wherein:

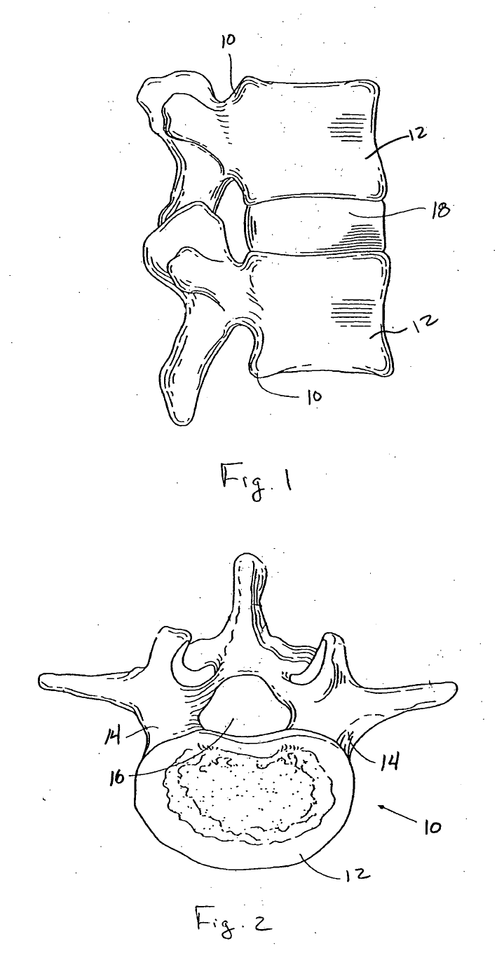

FIG. 1 is a side elevational view of a vertebral lumbar facet joint;

FIG. 2 is a superior view of a vertebra of shown in FIG. 1;

FIG. 3 is a front elevational view of a surgical implant in accordance with an embodiment of the present disclosure;

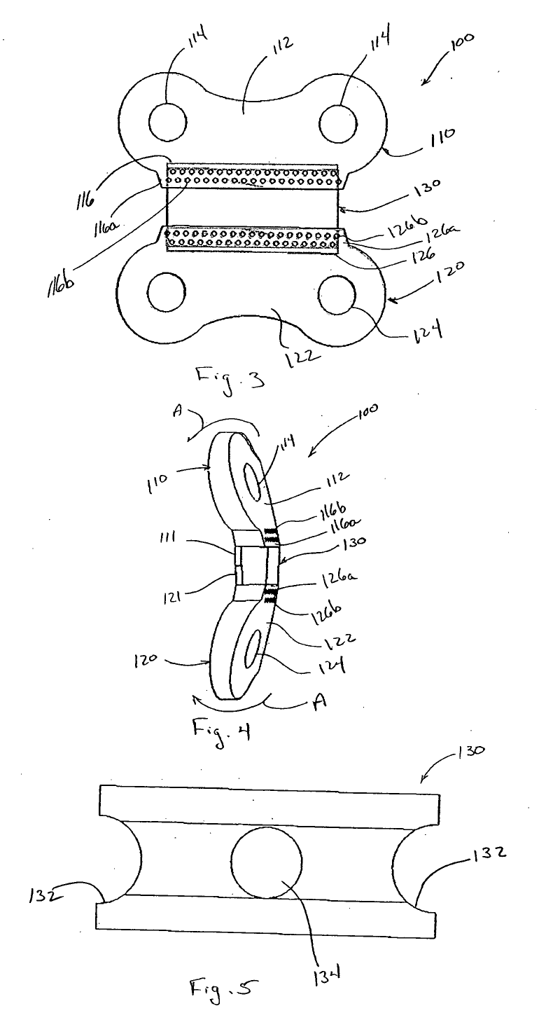

FIG. 4 is a side elevational view of the surgical implant of FIG. 3;

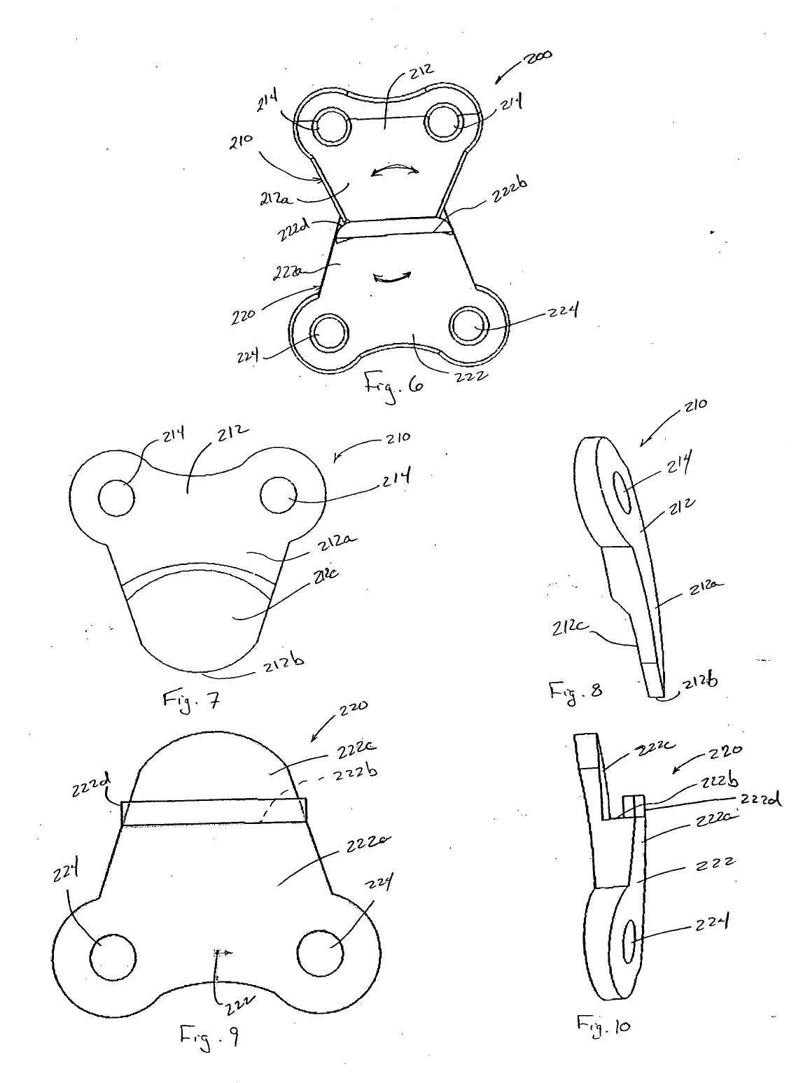

FIG. 5 is a top plan view of an insert for use with the surgical implant of FIGS. 3 and 4;

FIG. 6 is a front elevational view of a surgical implant according to another embodiment of the present disclosure;

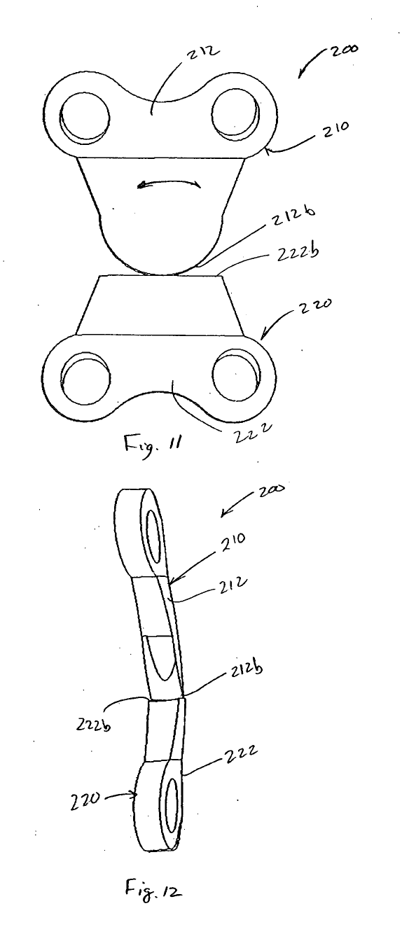

FIG. 7 is a front elevational view of a superior plate member of the surgical implant of FIG. 6;

FIG. 8 is a side elevational view of the superior plate member of FIG. 7;

FIG. 9 is a front elevational view of an inferior plate member of the surgical implant of FIG. 6;

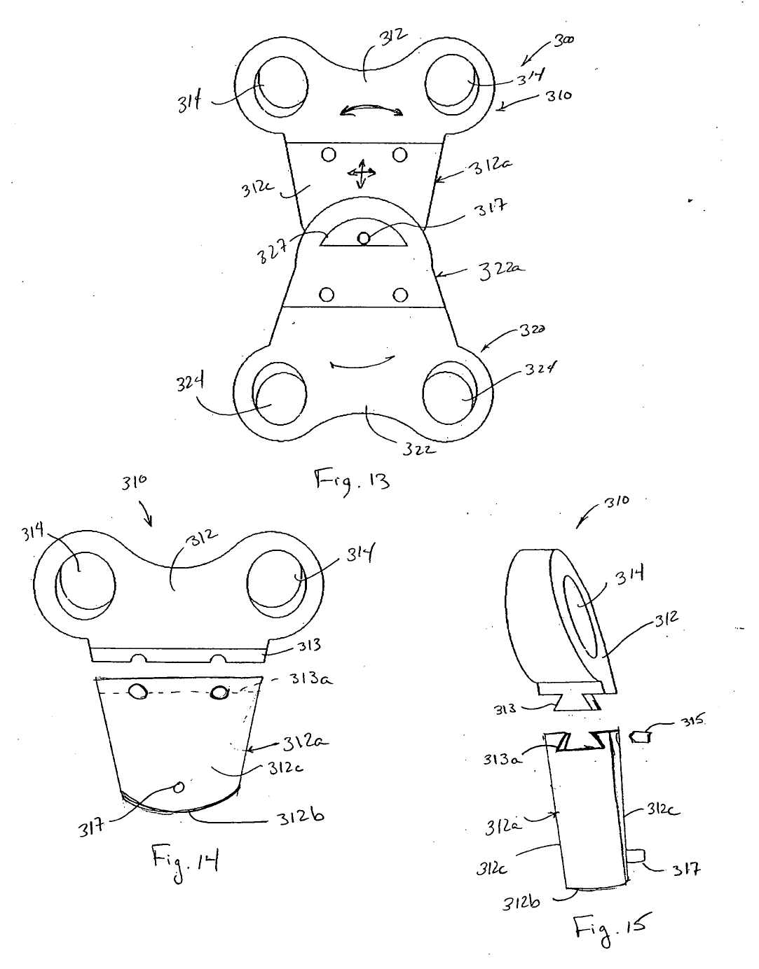

FIG. 10 is a side elevational view of the inferior plate member of FIG. 9;

FIG. 11 is a front elevational view of a surgical implant according to yet another embodiment of the present disclosure;

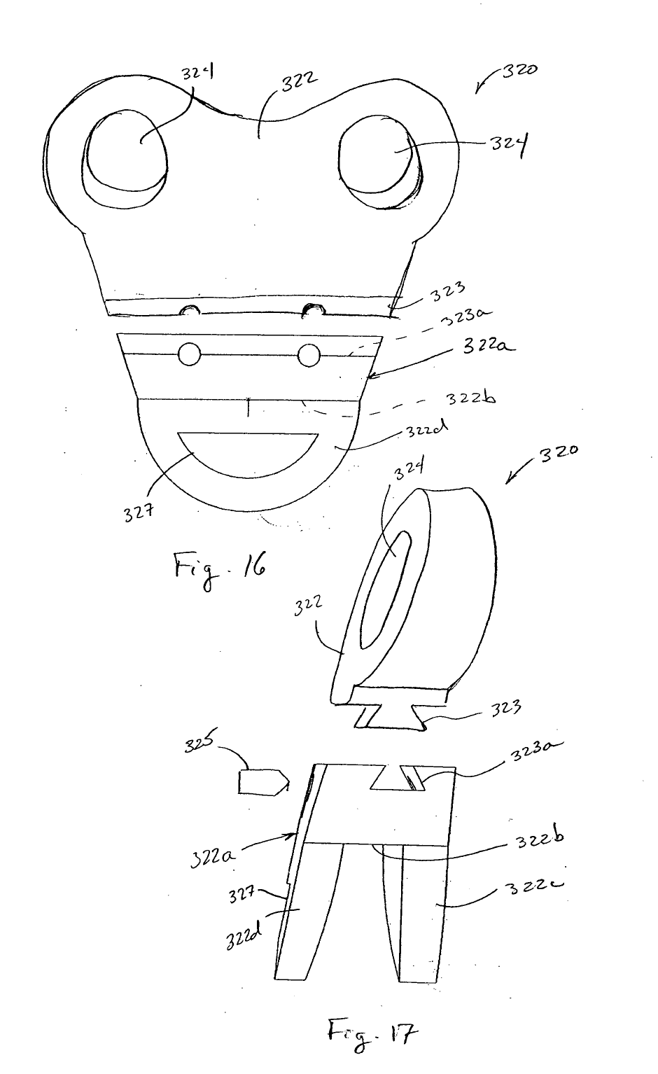

FIG. 12 is a side elevational view of the surgical implant of FIG. 11;

FIG. 13 is a front elevational view of a surgical implant according to a further embodiment of the present disclosure;

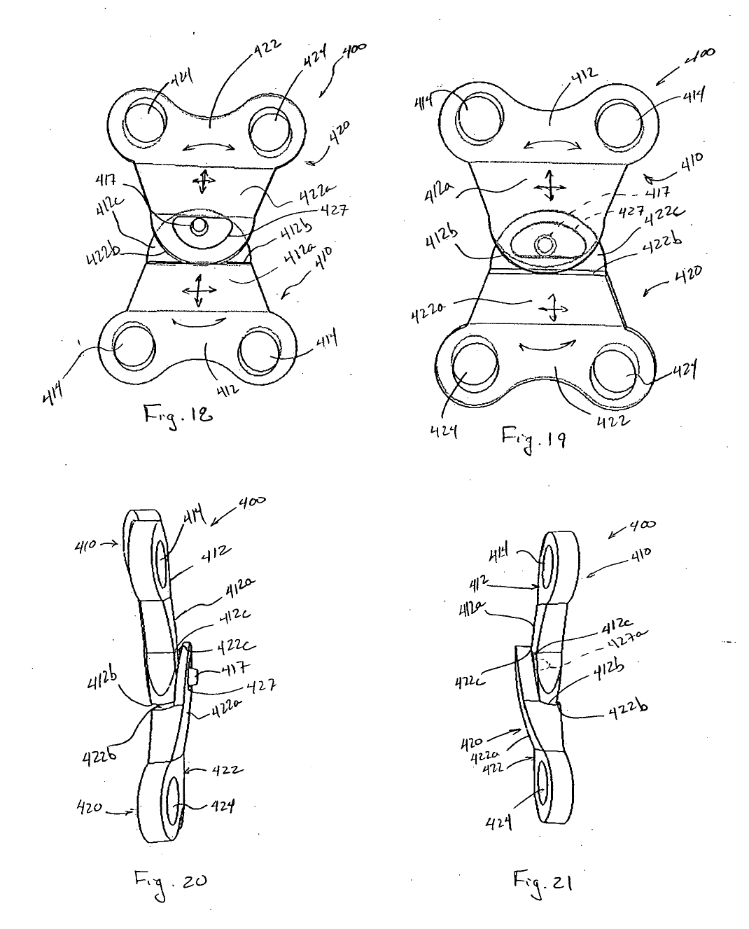

FIG. 14 is a front elevational view of a superior plate member, with parts separated, of the surgical implant of FIG. 13;

FIG. 15 is a side elevational view of the superior plate member of FIG. 14, shown with parts separated;

FIG. 16 is a front elevational view of an inferior plate member, with parts separated, of the surgical implant of FIG. 13;

FIG. 17 is a side elevational view of the inferior plate member of FIG. 16, shown with parts separated;

FIG. 18 is a front elevational view of a surgical implant according to still another embodiment of the present disclosure;

FIG. 19 is a rear elevational view of the surgical implant of FIG. 18;

FIG. 20 is a side elevational view of the surgical implant of FIG. 18 and 19;

FIG. 21 is a side elevational view of a variation of the surgical implant of FIGS. 18-20;

FIG. 22 is a front elevational view of the surgical implant of FIGS. 18-20, illustrating implant screws operatively associated therewith;

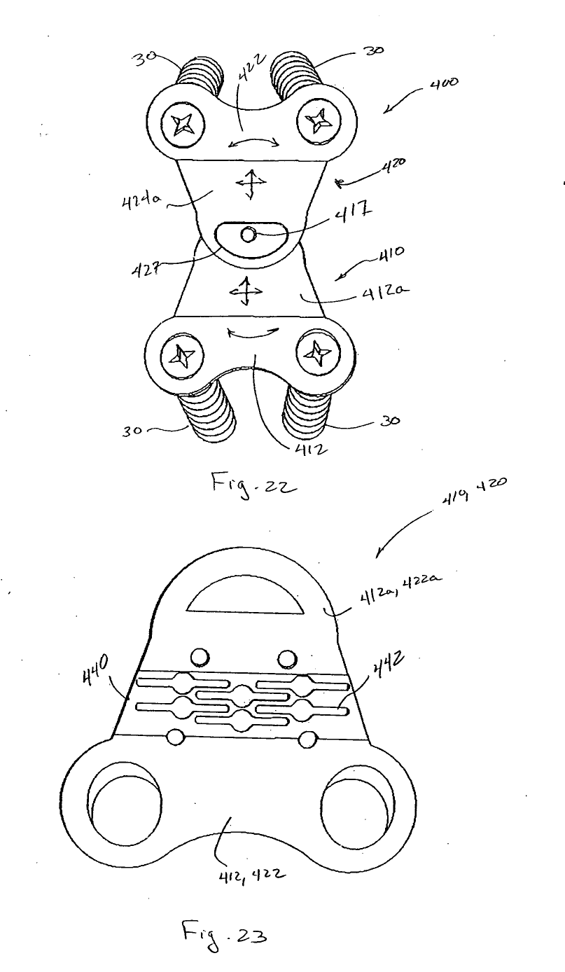

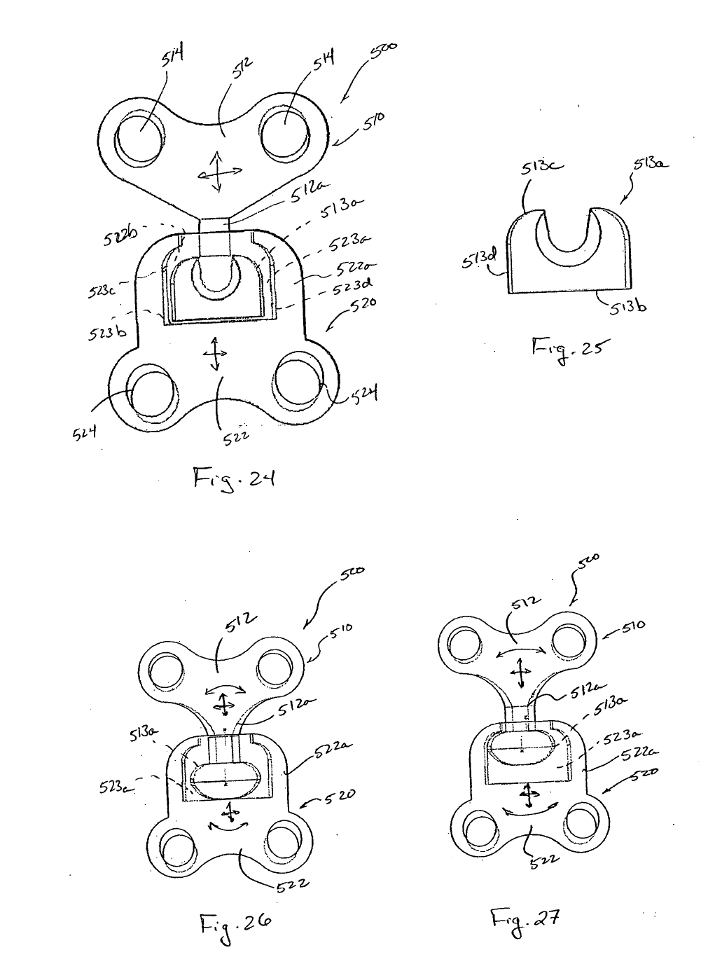

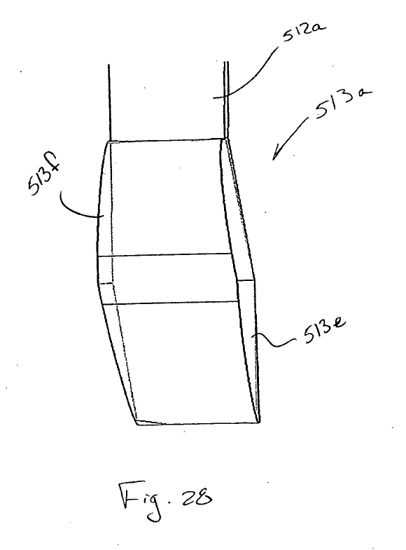

FIG. 23 is a front elevational view of a superior/inferior plate member of a surgical plate in accordance with an embodiment of the present disclosure;

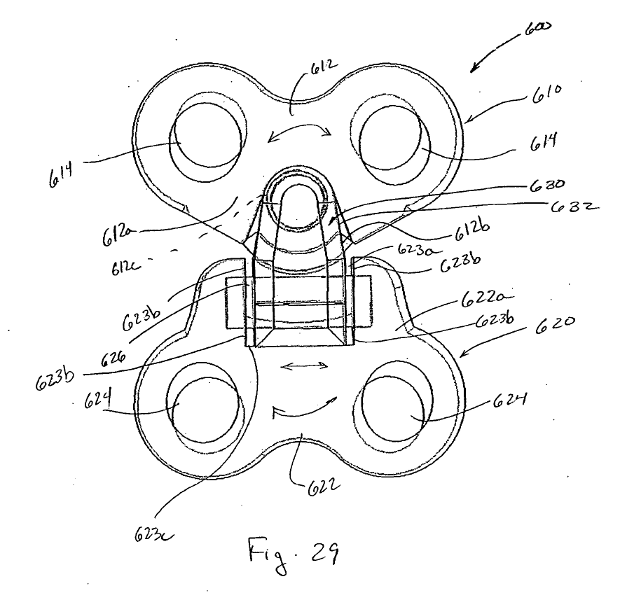

FIG. 24 is a front elevational view of a surgical implant according to yet another embodiment of the present disclosure;

FIG. 25 is a hub of the surgical implant of FIG. 24;

FIG. 26 is a front elevational view of a variation of the surgical implant of FIG. 24, shown in a first configuration;

FIG. 27 is a front elevational view of the surgical implant of FIG. 26, shown a second configuration;

FIG. 28 is an enlarged side elevational view of a head portion of the superior plate of the surgical plants of FIGS. 26 and 27;

FIG. 29 is a front elevational view of a surgical implant according to yet another embodiment of the present disclosure;

FIG. 30 is a front elevational view of a superior plate member of the surgical implant of FIG. 29;

FIG. 31 is a front elevational view of an inferior plate member of the implant of FIG. 29;

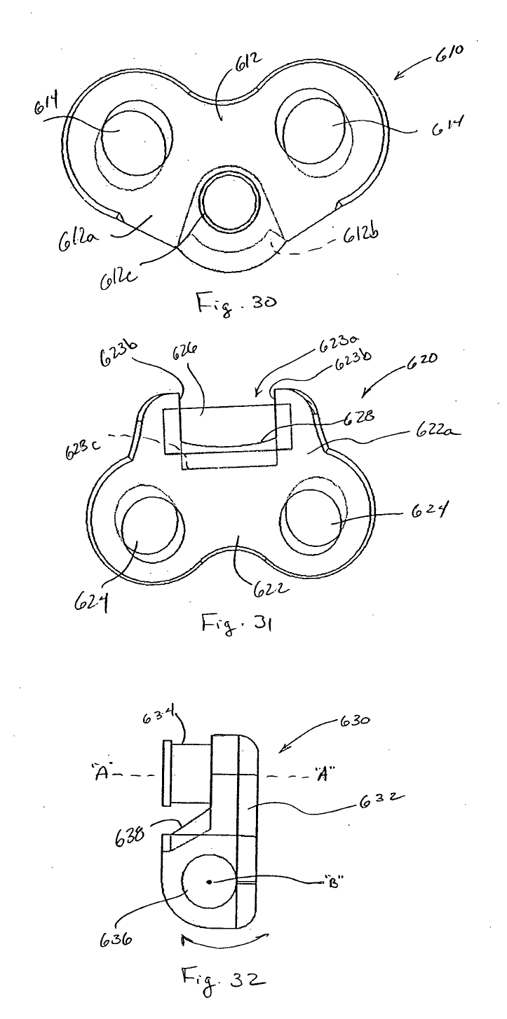

FIG. 32 is a side elevational view of a joint member for interconnecting the superior plate member and inferior plate member of the surgical implant of FIG. 29;

FIG. 33 is a front elevational view of a surgical implant according to another embodiment of the present disclosure;

FIG. 34 is a front elevational view of a superior/inferior plate member of a surgical implant according to an embodiment of the present disclosure;

FIG. 35 is a front elevational view of a surgical implant according to another embodiment of the present disclosure;

FIG. 36 is a rear elevational view of the surgical implant of FIG. 35;

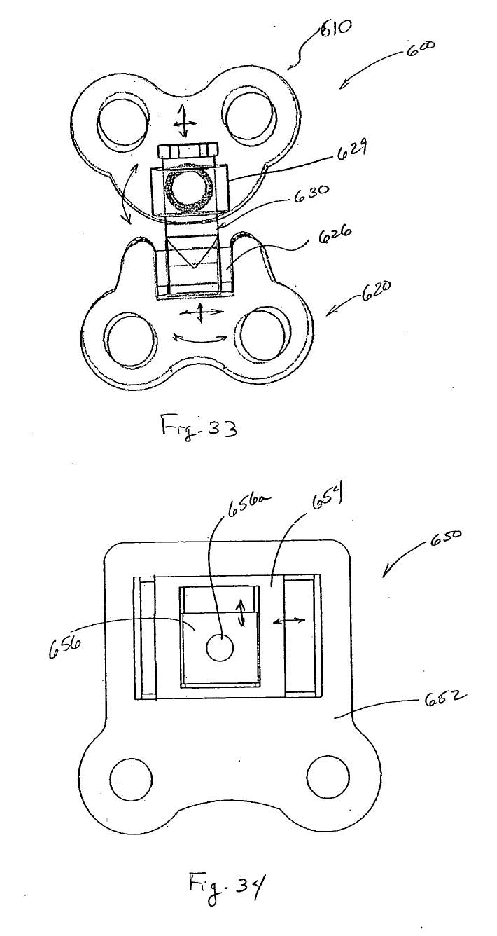

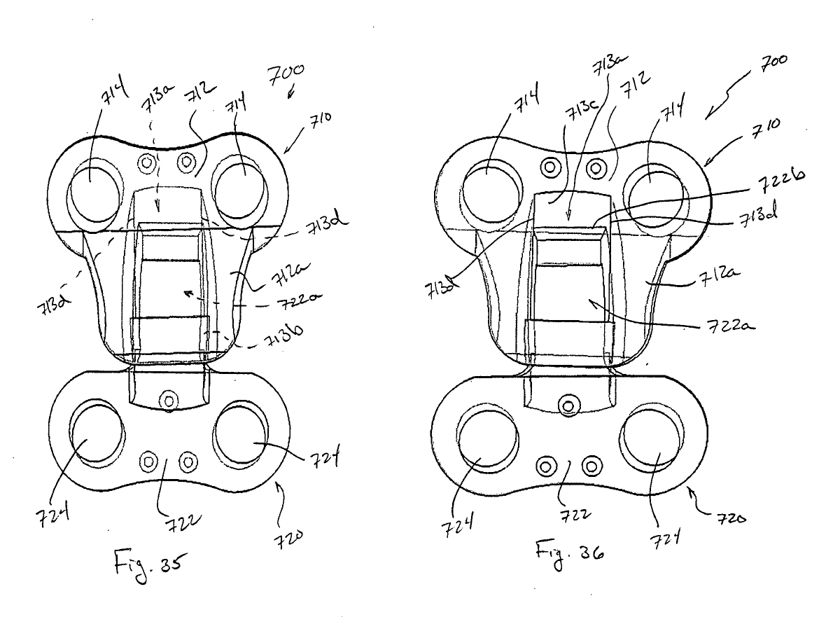

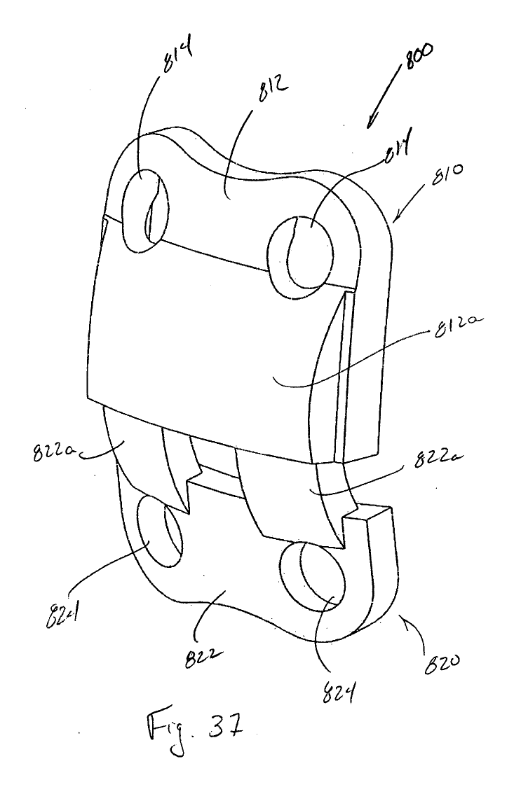

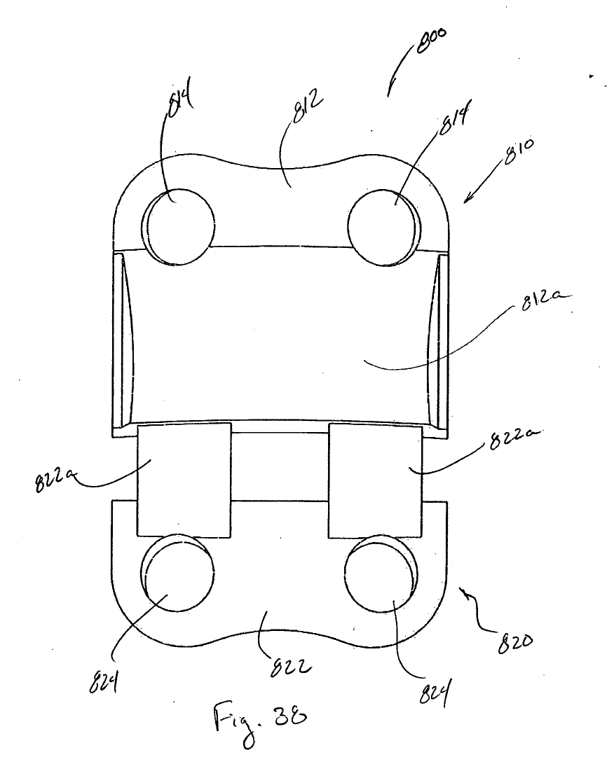

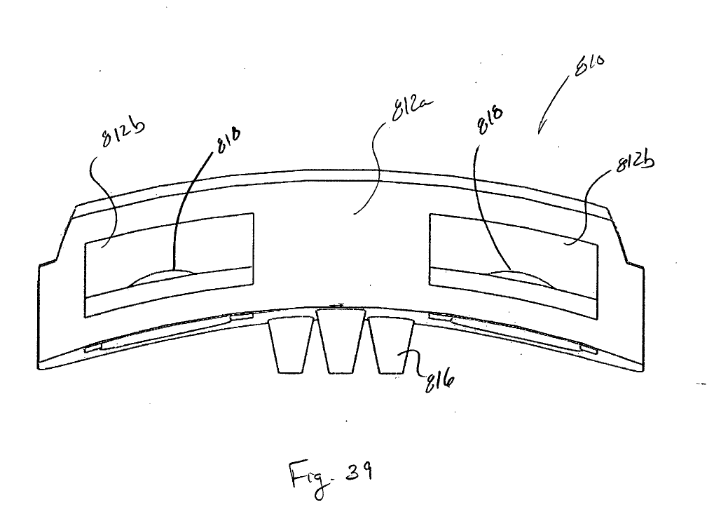

FIG. 37 is a perspective view of a surgical implant according to another embodiment of the present disclosure;

FIG. 38 is a front, elevational view of the surgical implant of FIG. 37; and

FIG. 39 is a bottom, plan view of a superior plate member of the surgical implant of FIGS. 37 and 38.

### DETAILED DESCRIPTION OF EMBODIMENTS

The present disclosure provides for novel surgical implants and alternate methods for treating the spine and eliminates many of the disadvantages previously described. The general principle of the present disclosure is to provide for dynamic stabilization devices and/or surgical implants for the spine that are fixed directly on or to vertebral bodies. Another feature of the present disclosure is that the devices and/or surgical implants allows for motion, movement or mobility in multiple directions. The devices and/or surgical implants of the present disclosure allow for rotation, flexion and lateral bending when implanted on the spine.

Various methods for achieving this motion are contemplated in this filing, however it is not the inventors' intention to limit the patents scope to those embodiments, but the general stabilization of the spine with allowance of multiple degrees of motion of the spine with an implant secured directly to the vertebral bodies of the spine.

Referring initially to FIG. 1, a pair of adjacent vertebra 10 are shown. As seen in FIGS. 1 and 2, each vertebra 10 includes a vertebral body 12, and a pair of pedicles 14 extending rearwardly of vertebral body 12 to form or define an arch 16. An intervertebral disc 18 interposed between each pair of adjacent vertebra 10 permits gliding movement of the vertebra 10 relative to one another thus permitting a range of movement of the vertebra 10 relative to one another.

Turning now to FIGS. 3-5, a surgical implant and/or plating system in accordance with an embodiment of the present disclosure, for selective or permanent fixation to a pair of adjacent vertebra 10, is generally designated as 100. As seen in FIGS. 3-5, surgical implant 100 includes a first or superior plate member 110, a second or inferior plate member 120, and a third or intermediate member 130 extending between and operatively inter-connecting superior and inferior plate members 110, 120.

As seen in FIGS. 3 and 4, superior plate member 110 and inferior plate member 120 are substantially identical to one another. Superior plate member 110 includes a plate-like body 112 having a substantially arcuate or concave profile. Body 112 defines at least one aperture 114 extending therethrough for receiving fastening members (not shown) used to fix or secure superior plate member 110 to a vertebra 10 (see FIGS. 1 and 2). Body 112 may include a recess or relief 116 formed in a side edge thereof wherein recess 116 is defined by a wall 116a extending along at least one side thereof.

Inferior plate member 120 includes a plate-like body 122 having a substantially arcuate or concave profile. Body 122 defines at least one aperture 124 extending therethrough for receiving fastening members (not shown) used to fix or secure inferior plate member 120 to a vertebra 10 (see FIGS. 1 and 2) adjacent to the vertebra to which superior plate member 110 is fixed. Body 122 may include a recess or relief 126 formed in a side edge thereof wherein recess 126 is defined by a wall 126a extending along at least one side thereof.

Superior plate member 110 and inferior plate member 120 may be fabricated from suitable surgically biocompatible and/or implantable materials, including and not limited to stainless steel, titanium and the like.

Intermediate portion 130 may be fabricated from a suitable elastomeric or metallic material, wherein intermediate portion 130 is adapted to permit relative movement of the superior and inferior plate members 110, 120. Intermediate portion 130 may be designed such that the shape thereof gives surgical implant 100 the motion desired. For instance, as seen in FIG. 5, intermediate portion 130 may include side cuts 132 that allow surgical implant 100 to bend laterally easier relative to a surgical implant including an intermediate portion 130 having no side cuts.

Intermediate portion 130 may include at least one aperture, hole or other feature formed therein. For example, as seen in FIG. 5, intermediate portion 130 may include a hole or lumen 134 formed in a center thereof, thereby allowing for greater compression of surgical implant 100.

In an embodiment, as seen in FIG. 4, at least one of superior plate member 110 and inferior plate member 120 includes a stop member (respective stop members 111, 121 shown in FIG. 4) which is configured to prevent the spine from bending backwards or extending, when surgical implant 100 is in place. In particular, stop members 111, 121 of respective superior and inferior plate members 110, 120 extend from a posterior surface thereof such that when surgical implant 100 is bent in a posterior direction, as indicated by arrows “A” of FIG. 4, stop members 111, 121 come into contact with one another and prevent further bending or movement of surgical implant 100 in the posterior direction. It is contemplated that stop members 111, 121 of respective superior and inferior plate members 110, 120 do not prevent surgical implant 100 from being bent in an anterior direction, opposite to the direction of arrows “A” of FIG. 4.

As seen in FIGS. 3 and 4, each of superior plate member 110 and inferior plate member 120 may include a plurality of holes 116b, 126b respectively, formed therein. In use, during the manufacture of surgical implant 100, elastomeric material of intermediate portion 130 may be molded into each of superior plate member 110 and inferior plate member 120, with polymer permeating into holes 116b, 126b. It is contemplated that other methods may be used as well.

It is contemplated that intermediate portion 130 may be affixed to only one of superior plate member 110 and inferior plate member 130. In this configuration, the elastomeric material of intermediate portion 130 would act as a bumper and would not put the elastomeric material in tension.

Turning now to FIGS. 6-10, a surgical implant and/or plating system according to another embodiment of the present disclosure, for selective or permanent fixation to a pair of adjacent vertebra 10, is generally designated as 200. Surgical implant 200 includes a first or superior plate member 210, and a second or inferior plate member 220 in juxtaposed relation to superior plate member 210 and operatively associated therewith.

As seen in FIGS. 6-8, superior plate member 210 includes a plate-like body 212 having a substantially arcuate or concave profile. Body 212 defines at least one aperture 214 extending therethrough for receiving fastening members (not shown) used to fix or secure superior plate member 210 to a vertebra 10 (see FIGS. 1 and 2). Body 212 defines a longitudinal plane and includes a tongue portion 212a extending substantially in the direction of the longitudinal plane. Body 212 defines a first bearing surface 212b oriented substantially perpendicular to the longitudinal plane of body 212 and a second bearing surface 212c oriented substantially parallel to the longitudinal plane.

Inferior plate member 220 includes a plate-like body 222 having a substantially arcuate or concave profile. Body 222 defines at least one aperture 224 extending therethrough for receiving fastening members (not shown) used to fix or secure inferior plate member 220 to a vertebra 10 (see FIGS. 1 and 2) adjacent to the vertebra to which superior plate member 210 is fixed. Body 222 defines a longitudinal plane and includes a tongue portion 222a extending substantially in the direction of the longitudinal plane. Body 222 defines a first bearing surface 222b oriented substantially perpendicular to the longitudinal plane of body 222 and a second bearing surface 222c oriented substantially parallel to the longitudinal plane. Body 222 may include a retaining wall 222d extending parallel to second bearing surface 222c and spaced a distance therefrom to define a space configured to receive an end of second bearing surface 212c of superior plate member 210 therein. Retaining wall 222d functions to maintain superior plate member 210 and inferior plate member 220 in substantially the same plane as one another.

Surgical implant 200 enables the surgeon to distract the spine and maintain the distraction. In particular, in use, with a two portion surgical implant like surgical implant 200, superior plate member 210 would be fixed to a vertebra and inferior plate member 220 would be fixed to an adjacent vertebra, although they need not be immediately adjacent. Superior plate member 210 and inferior plate member 220 are designed to bear against each other, thereby maintaining the distraction of the spine, but allowing motion along the bearing surface. In particular, first bearing surface 212b of superior plate member 210 is configured to bear or contact against first bearing surface 222b of inferior plate member 220, and second bearing surface 212c of superior plate member 210 is configured to bear or contact against second bearing surface 222c of inferior plate member 220.

The inter-engagement of first bearing surfaces 212b and 222b of respective superior and inferior plate members 210, 220 prevents distraction of the spine from relaxing. The inter-engagement of second bearing surfaces 212c and 222c of respective superior and inferior plate members 210, 220 allows/permits rotation, extension and lateral bending of superior plate member 210 relative to inferior plate member 220 and thus of the spine.

Bearing surface 212c, 222c may have a substantially cylindrical or spherical profile. In an embodiment, the profile of bearing surface 212c and/or bearing surface 222c may be substantially spherical such that the motion allowed places the axis of rotation as close as possible to the natural axis. This can be done by fixing the radius of the sphere and its mid-point relative to the plate member equal to the natural axis of rotation. For instance, if the axis of rotation is in the superior plate member of the inferior vertebra, and approximately 25 mm from the surface of the anterior surface of the vertebra, then the sphere could be designed with a radius of 27 mm and the center of the sphere 5 mm below the midpoint of the superior plate member. The 27 mm is selected because it is the sum of the 25 mm distance from the axis to the vertebral wall and 2 mm to allow for the thickness of the surgical implant. The 5 mm is below the midpoint and assumes a 10 mm disc height and placement of the midpoint of the plate member at the mid point of the disc. Using this, the bearing surface 212c or 222c will be only a fraction of the total sphere.

It is contemplated that each of superior plate member 210 and inferior plate member 220 may be separated into two or more pieces, such that when assembled they form a complete plate member. This may be required or desirable for a number of reasons. For instance, titanium is often used as an implant material because of its excellent biocompatibility and strength, therefore may be desirable for that piece which is to be fixed to the vertebra. However, titanium is a poor material for defining a bearing surface and thus other materials may be better suited as defining the bearing surface to reduced wear and wear debris. It may be more desirable to have the bearing surface made out of cobalt chrome steel or the like. In this case the cobalt chrome can be polished to provide a low wear bearing surface for the articulation.

In an alternate embodiment, superior and/or inferior plate members 210, 220 may be constructed so as to include a core of titanium covered by a shell of cobalt chrome, or a cobalt chrome liner may be placed inside of pocket defined in a titanium implant. The two materials may be secured or affixed to one another by means of press fitting or the like.

Turning now to FIGS. 11 and 12, surgical implant 200 has been modified to remove second bearing surface 222c and retaining wall 222d of inferior plate member 220. Accordingly, as shown in FIGS. 11 and 12, first bearing surface 212b of superior plate member 210 engages or contacts against first bearing surface 222b of inferior portion 222.

As seen in FIGS. 11 and 12, surgical implant 200 may only be provided with a single bearing surface on a minor dimension of the superior plate member 210 and/or inferior plate member 220. Such a surgical implant 200, as shown in FIGS. 11 and 12, may be used when a surgeon only desires to hold a distraction of the vertebra or spine, but not limit motion in any other direction of the vertebra or spine. Such a surgical implant could be referred to as a compression stop, although the surgical implant would tend to maintain the distraction more on the side of the vertebra to which it is affixed. In order to overcome this limitation, a stop (not shown, similar to stops 111, 121 of surgical implant 100) may be provided which would prevent superior plate member 210 and inferior plate member 220 of surgical implant 200 from tilting while the bearing surfaces 212b, 222b thereof maintain the distraction, thereby eliminating or reducing the tendency for surgical implant to distract more on the side of application.

As seen in FIGS. 7, 8, 11 and 12, first bearing surface 212b of superior plate member 210 defines a substantially arcuate or curved profile, thereby allowing for superior plate member 210 to roll on first bearing surface 222b of inferior plate member 220.

Turning now to FIGS. 13-17, a surgical implant and/or plating system according to another embodiment of the present disclosure, for selective or permanent fixation to a pair of adjacent vertebra 10, is generally designated as 300. Surgical implant 300 includes a first or superior plate member 310, and a second or inferior plate member 320 in juxtaposed relation to superior plate member 310 and operatively associated therewith.

As seen in FIGS. 13-15, superior plate member 310 includes a plate-like body 312 having a substantially arcuate or concave profile. Body 312 defines at least one aperture 314 extending therethrough for receiving fastening members (not shown) used to fix or secure superior plate member 310 to a vertebra 10 (see FIGS. 1 and 2). Body 312 defines a longitudinal plane and includes a tongue portion 312a extending substantially in the direction of the longitudinal plane. Body 312 defines a first bearing surface 312b oriented substantially perpendicular to the longitudinal plane of body 312 and at least a second bearing surface 312c oriented substantially parallel to the longitudinal plane.

As seen in FIGS. 14 and 15, superior plate member 310 may be separated into at least a pair of pieces wherein body 312 comprises one piece and tongue portion 312a comprises the second piece. Body 312 and tongue portion 312a may be selectively connectable to one another via respective cooperating complementary engaging structure 313, 313a, such as, for example, dove-tail type joints or any other suitable mechanical joint structure. Body 312 and tongue portion 312a may be secured to one another via suitable fastening devices 315 such as, for example set screws or the like extending through engaging structure 313a of tongue portion 312a and contacting against engaging structure 313 of body 312.

Inferior plate member 320 includes a plate-like body 322 having a substantially arcuate or concave profile. Body 322 defines at least one aperture 324 extending therethrough for receiving fastening members (not shown) used to fix or secure inferior plate member 320 to a vertebra 10 (see FIGS. 1 and 2) adjacent to the vertebra to which superior plate member 310 is fixed. Body 322 defines a longitudinal plane and includes a tongue portion 322a extending substantially in the direction of the longitudinal plane. Body 322 defines a first bearing surface 322b oriented substantially perpendicular to the longitudinal plane of body 322 and at least a second bearing surface 322c oriented substantially parallel to the longitudinal plane. Body 222 may include a retaining wall 322d extending substantially parallel to second bearing surface 322c and spaced a distance therefrom to define a space configured to receive an end of second bearing surface 312c of superior plate member 312 therein. Retaining wall 322d functions to maintain superior plate member 310 and inferior plate member 320 in substantially the same plane as one another.

As seen in FIGS. 16 and 17, inferior plate member 320 may be separated into at least a pair of pieces wherein body 322 comprises one piece and tongue portion 322a comprises the second piece. Body 322 and tongue portion 322a may be selectively connectable to one another via respective cooperating complementary engaging structure 323, 323a, such as, for example, dove-tail type joints or any other suitable mechanical joint structure. Body 322 and tongue portion 322a may be secured to one another via suitable fastening devices 325 such as, for example set screws or the like extending through engaging structure 323a of tongue portion 322 and contacting against engaging structure 323 of body 322.

As seen in FIGS. 13-15, superior plate member 310 may include a guide pin 317 extending from a surface of tongue portion 312a. As seen in FIGS. 13, 16 and 17, inferior plate member 320 may include a window, aperture or recess 327 formed in retaining wall 322d of tongue portion 322a into which guide pin 317 extends or projects. Window 327 defines an area in which guide pin 317 may travel and limits the movement of superior plate member 310 relative to inferior plate member 320 based on the dimensions and configuration of guide pin 317 and window 327.

Turning now to FIGS. 18-22, a surgical implant and/or plating system according to another embodiment of the present disclosure, for selective or permanent fixation to a pair of adjacent vertebra 10, is generally designated as 400. Surgical implant 400 includes a first or superior plate member 410, and a second or inferior plate member 420 in juxtaposed relation to superior plate member 410 and operatively associated therewith.

As seen in FIGS. 18-22, superior plate member 410 includes a plate-like body 412 having a substantially arcuate or concave profile. Body 412 defines at least one aperture 414 extending therethrough for receiving fastening members 30 (see FIG. 22) used to fix or secure superior plate member 410 to a vertebra 10 (see FIGS. 1 and 2). Body 412 defines a longitudinal plane and includes a tongue portion 412a extending substantially in the direction of the longitudinal plane. Body 412 defines a first bearing surface 412b oriented substantially perpendicular to the longitudinal plane of body 412 and at least a second bearing surface 412c oriented substantially parallel to the longitudinal plane.

Inferior plate member 420 includes a plate-like body 422 having a substantially arcuate or concave profile. Body 422 defines at least one aperture 424 extending therethrough for receiving fastening members 30 (see FIG. 22) used to fix or secure inferior plate member 420 to a vertebra 10 (see FIGS. 1 and 2) adjacent to the vertebra to which superior plate member 410 is fixed. Body 422 defines a longitudinal plane and includes a tongue portion 422a extending substantially in the direction of the longitudinal plane. Body 422 defines a first bearing surface 422b oriented substantially perpendicular to the longitudinal plane of body 422 and at least a second bearing surface 422c oriented substantially parallel to the longitudinal plane.

As seen in FIGS. 18-22, surgical implant 400 has a main bearing surface formed on a minor cross-section of the superior plate member 410 and/or inferior plate member 420 in order to maintain the distraction. As seen in FIGS. 18-22, bearing surface 422c of inferior plate member 420 is designed to stop motion at certain limits. While bearing surface 422c is shown as an extension of body 422 of inferior plate member 420, it is contemplated that body 412 of superior plate member 410 may include a suitable extension projecting therefrom. In an embodiment, the extension of second bearing surface 422c may be angled away from body 422 of inferior plate member 420 and as shown, the extension of second bearing surface 422c would limit extension when in the neutral position, while the bearing surfaces 412b, 422b are in contact. However, the extension of second bearing surface 422c may permit flexion to the degree that the extension of second bearing surface 422c angles away from body 412 of inferior plate member 420.

As seen in FIG. 20, superior plate member 410 is capable of tilting forward by the angle that second bearing surface 422c of inferior plate member 420 is relieved from tongue portion 412a of superior plate member 410. Superior plate member 410 is prevented from tilting backward relative to second bearing surface 422c of inferior plate member 420 because the relief therebetween is angular, i.e., at the interface between bearing surface 422b and bearing surface 412b, the relief between second bearing surface 412c of superior plate member 410 and second bearing surface 422c of inferior plate member 420 is approximately equal to zero (0) mm. At a location near an end of second bearing surface 422c of inferior plate member 420 the relief between second bearing surface 412c of superior plate member 410 and second bearing surface 422c of inferior plate member 420 is approximately equal to 0.5 mm.

As seen in FIGS. 18-20 and 22, superior plate member 410 may include a guide pin 417 extending from a surface of tongue portion 412a, and inferior plate member 420 may include a window, aperture or recess 427 formed in retaining wall second bearing wall 422c of tongue portion 422a into which guide pin 417 extends or projects. Window 427 defines an area in which guide pin 417 may travel and limits the movement of superior plate member 410 relative to inferior plate member 420 based on the dimensions and configuration of guide pin 417 and window 427. In an alternate embodiment, as seen in FIG. 21, a guide pin 427a may extend from second bearing wall 422c of tongue portion 422a of inferior plate member 420 and into a window (not shown) formed in tongue portion 412a of superior plate member 410.

In use, the relative motion of superior plate member 410 and inferior plate member 420 is limited by the edges of window 427. The motion may be regulated by determining the center of rotation of the spinal level to be fixed and the degree of angulation to be allowed. By use of simple trigonometry, the length and height of window 427 may be determined. For example, if the center of the spine rotation is 17 mm from surgical implant 400 and 10° of angulation is desired, then 17 times the sine of 10 mm of clearance must be provided for pin 417 in window 427. In the instant example, 10° of motion would require approximately 3 mm of clearance for pin 417. As mentioned above, pin 417 may be placed on either plate member 410, 420 and window 427 formed in the other plate member 410, 420. It is further contemplated that guide pin 417 may extend toward the spine, as shown in FIG. 21.

As seen in FIG. 23, superior plate member 410 and/or inferior plate member 420 may include an insert 440 interposed between respective body 412, 422 and respective tongue portion 412a, 422a. It is contemplated that bodies 412, 422 of superior/inferior plate members 410, 420 may be fabricated from titanium, tongue portions 412a, 422a of superior/inferior plate members 410, 420 may be fabricated from cobalt chrome, and insert 440 may be fabricated from nitinol (e.g., Nickel-Titanium Alloy). It is further contemplated that insert 440 may be manufactured to be flexible under load, thereby allowing a surgical implant including such an insert to compress under said loads and create a dynamic stabilization thereof. As seen in FIG. 23, insert 440 may include at least one aperture 442 formed therein and/or therethrough, wherein the apertures 442 compress upon exposure to a load. It is envisioned that apertures 442 may be in the form of elongate slots having relatively enlarged central portions, or any other suitable configuration, contemplated by one having skill in the art, for the intended purpose of providing dynamic stabilization.

Turning now to FIGS. 24 and 25, a surgical implant and/or plating system according to another embodiment of the present disclosure, for selective or permanent fixation to a pair of adjacent vertebra 10, is generally designated as 500. Surgical implant 500 includes a first or superior plate member 510, and a second or inferior plate member 520 in juxtaposed relation to superior plate member 510 and operatively associated therewith.

As seen in FIGS. 24 and 25, superior plate member 510 includes a plate-like body 512 which may have a substantially arcuate or concave profile. Body 512 defines at least one aperture 514 extending therethrough for receiving fastening members (not shown) used to fix or secure superior plate member 510 to a vertebra 10 (see FIGS. 1 and 2).

Body 512 defines a longitudinal plane and includes a tongue or stem portion 512a extending substantially in the direction of the longitudinal plane. A distal end of stem portion 512a is configured to operatively engage a head member 513a, wherein the head member 513a had a transverse cross-sectional profile which is larger than a transverse cross-sectional profile of stem portion 512a. Head member 513a may be pivotably and/or rotatably connected to the distal end of stem portion 512a. Head member 513a may define a plurality of bearing surfaces including, and not limited to, a bottom bearing surface 513b, a top bearing surface 513c, and a side bearing surface 513d.

Inferior plate member 520 includes a plate-like body 522 having a substantially arcuate or concave profile. Body 522 defines at least one aperture 524 extending therethrough for receiving fastening members (not shown) used to fix or secure inferior plate member 520 to a vertebra 10 (see FIGS. 1 and 2) adjacent to the vertebra to which superior plate member 510 is fixed. Body 522 defines a longitudinal plane and includes a tongue portion 522a extending substantially in the direction of the longitudinal plane.

Tongue portion 522a of inferior plate member 520 defines a recess or hollow 523a formed in a side surface thereof, in juxtaposed relation to stem portion 513a. Hollow 523a is further defined by a rim 522b extending at least partially around an opening thereof. Hollow 523a is configured to receive head portion 513a of superior plate member 510 therein. Hollow 523a may define a plurality of bearing surfaces including, and not limited to, a bottom bearing surface 523b, a top bearing surface 523c defined by rim 522b, and side bearing surfaces 523d.

As seen in FIG. 25, head portion 513a defines a cut-out 513e, having a substantially spherical profile, that is configured to mate with the end of stem portion 512a, which may also have a spherical profile.

In an alternate embodiment, as seen in FIGS. 26 and 27, head portion 513a of superior plate member 510 may be fixedly secured or integrally formed with stem portion 512a. Head portions 513a may have substantially circular or cylindrical cross-sectional profiles.

An entrance to hollow 523a may be located on the minor cross-section of inferior plate member 520, such that inferior plate member 520 does not extend much off the surface of the vertebral body, or has a substantially low profile, when attached. Hollow 523a and inferior plate member 520 may be designed such that head portion 513a may only fit in the entrance to hollow 523a when twisted in a motion or direction that surgical implant 500 would not be exposed to when in use in the body. In this manner, in use, superior and inferior plate members 510, 520 of surgical implant 500 will be locked together and allow a limited range of motion of surgical implant 500 and of the spine.

In an embodiment, hollow 523a may be about 2 mm to 4 mm larger on each side thereof than head portion 513a. It is contemplated that a thickness, depth or width of hollow 523a is substantially similar to a thickness of head portion 513a. In this manner, each plate member 510, 520 of surgical implant 500 is allowed to move relative to each other by an amount equal to approximately 2 mm to 4 mm, when implanted. This in turn will allow a superior vertebral body and inferior vertebral body, to which surgical implant 500 is fixed, to move relative to one another by an amount equal to approximately 2 mm to 4 mm, in a plane of the cavity. Since the thickness of head portion 513a and of hollow 523a are substantially similar, the vertebral bodies will not be allowed to move in this plane.

When surgical implant 500 is implanted as intended, surgical implant 500 prevents the vertebral bodies from translating. The open space between hollow 523a and head portion 513a allows for the vertebral bodies to rotate, flex and extend, and bend laterally. The shapes of the mating parts may be changed or modified as needed to allow or disallow various types of motion. For instance, if the mating or bearing surfaces of head portion 513a and hollow 523a are cylindrical then lateral bending will be eliminated, but rotation will be allowed. If the mating or bearing surfaces of head portion 513a and hollow 523a are spherical, then each of the motions mentioned above would be permitted.

The spacing between head portion 513a and hollow 523a is determined by selecting about which point in space surgical implant 500 should rotate or pivot. In one instance, the rotation point may be selected to be approximately 17.5 mm behind surgical implant 500 from a middle of surgical implant 500. The desired motion of the two vertebral bodies is then determined. In the instance case, approximately 10° in rotation, flex and lateral bending was selected. Given the value of a desired angle of movement to allow and a value of a radius of rotation, an amount of space required, between head portion 513a and hollow 523a, for the desired motion may be calculated using simple trigonometry.

It is contemplated that a part or portion of a bearing surface of head portion 513a may be recessed to allow surgical implant 500 to move in a third dimension that is not allowed by the space in hollow 512a. Further, hollow 523a may be made to allow for movement in only a single direction in the third plane, either forwards or backwards. To accomplish this, each side of the bearing surface of head portion 513a must be recessed on opposite sides of its middle access.

In one embodiment, surgical implants 500 are designed for placement on the anterior part of the cervical spine. To facilitate the geometry of the vertebral bodies superior/inferior plate members 510, 520 of surgical implant 500 each have a convex side and a concave side. In an embodiment, superior/inferior plate members 510, 520 may be constructed to permit superior/inferior plate members 510, 520 to bend towards its convexity, but not its concavity. FIG. 28 illustrates the cuts required to allow this motion.

As seen in FIG. 28, a lower front surface 513e of head portion 513a is substantially parallel to an upper rear surface 513f of head portion 513a. Head portion 513a has substantially the same thickness as the cavity or hollow into which it is to be received. As such, an upper half section of head portion 513a is not capable of moving to the left of the figure and a lower half section of head portion 513a is not capable of moving to the right of the figure. However, the sides opposite to lower front surface 513e and upper rear surface 513f of head portion 513a is relieved relative to the surfaces of the hollow or cavity. In this manner, the upper half section of head portion 513a and the lower half section of head portion 513b are each capable of rotating in the direction of their respective relief's.

FIGS. 26 and 27 illustrate how superior/inferior plate members 510, 520 move relative to each other. It is contemplated that the excess space in hollow 523a, e.g., the space between head portion 513a and the walls of hollow 523a, may be filled with polymer, gel or foam to dampen the motion. A plastic film (not shown) may also be draped around the smaller cross-section of the superior plate member 510 and extend over hollow 523a of inferior plate member 520. This could be done to either trap wear debris or to contain a dampening fluid or gel within hollow 523a.

Turning now to FIGS. 29-32, a surgical implant and/or plating system according to another embodiment of the present disclosure, for selective or permanent fixation to a pair of adjacent vertebra 10, is generally designated as 600. Surgical implant 600 includes a first or superior plate member 610, and a second or inferior plate member 620 in juxtaposed relation to superior plate member 610 and operatively associated therewith.

As seen in FIGS. 29 and 30, superior plate member 610 includes a plate-like body 612 which may have a substantially arcuate or concave profile. Body 612 defines at least one aperture 614 extending therethrough for receiving fastening members (not shown) used to fix or secure superior plate member 610 to a vertebra 10 (see FIGS. 1 and 2).

Body 612 defines a longitudinal plane and includes a tongue portion 612a extending substantially in the direction of the longitudinal plane. Tongue portion 612a defines a cut-out or recess 612b formed in a surface thereof, and an aperture 612c formed therein, wherein aperture 612c may be formed in cut-out 612b. Cut-out 612b may be defined by a pair of side walls which may be parallel to one another, or as shown in FIGS. 29 and 30, which may be angled with respect to one another, such that longitudinal axes thereof intersect at a location above aperture 612c, as viewed in FIGS. 29 and 30.

As seen in FIGS. 29 and 31, inferior plate member 620 includes a plate-like body 622 having a substantially arcuate or concave profile. Body 622 defines at least one aperture 624 extending therethrough for receiving fastening members (not shown) used to fix or secure inferior plate member 620 to a vertebra 10 (see FIGS. 1 and 2) adjacent to the vertebra to which superior plate member 610 is fixed. Body 622 defines a longitudinal plane and includes a tongue portion 622a extending substantially in the direction of the longitudinal plane.

Tongue portion 622a of inferior plate member 620 defines a cut-out 623a formed in a side surface thereof and extending completely therethrough. Cut-out 623a is defined by a pair of spaced apart, juxtaposed side walls 623b and a base wall 623c. Inferior plate member 620 further includes a pin or rod 626 extending across and between side walls 623b. Rod 626 is spaced a distance from base wall 623c.

As seen in FIGS. 29 and 32, surgical implant 600 further includes an inter-engagement member 630 operatively connected to each of superior plate member 610 and inferior plate member 620. Inter-engagement member 630 includes a body portion 632, a nub of boss 634 extending from a side surface of body portion 632 proximate a first end thereof, and a lumen 636 extending through body portion 632 proximate a second end thereof. A longitudinal axis “A” of boss 634 is oriented substantially orthogonal to a longitudinal axis “B” of lumen 636.

Boss 634 is configured and dimensioned for rotatable engagement in aperture 612c of superior plate member 610, and lumen 636 is configured and dimensioned for slidable and rotatable engagement or support on rod 626 of inferior plate member 620.

Body portion 632 of inter-engagement member 630 is dimensioned so as to have a width which is equal to or less than a dimension between side walls 623d of cut-out 623a of inferior plate member 620. If body portion 632 has a width that is less than the dimension between side walls 623d of cut-out 623a of inferior plate member 620, in use, body portion 632 of inter-engagement member 630 may slide along a length of rod 626.

As so coupled, superior and inferior plate members 610, 620 are capable of rotation relative to one another, however, only to as much as is allowed by a space defined between inter-engagement member 630 and opposed surfaces 623b of cut-out 623a of inferior plate members 620. To allow approximately 10° of rotation with approximately a 17.5 mm axis of rotation, approximately 3 mm of space must be allowed.

Inter-engagement member 630 may also rotate about rod 626 to allow the vertebral bodies to flex and extend. Flexion or extension may be selectively eliminated by placing a stop feature 628 on inferior plate member 620 and/or a stop feature 638 on body portion 632 of inter-engagement member 630. In this manner, when superior plate member 610, inferior plate member 620 and inter-engagement member 630 rotate to a specific position, stops 628, 638 block further movement thereof.

Turning now to FIG. 33, a variation of surgical implant 600 is shown. As seen in FIG. 33, inter-engagement member 630 may be in the form of a rod or cylinder and is generally designated as 630a. A first end of inter-engagement member 630a may slidably extend through a hub 629 pivotably connected to superior plate member 610, and a second end of inter-engagement member 630a may slidably and rotatably engage rod 626 of inferior plate member 620 in a manner substantially similar to inter-engagement member 630.

Turning now to FIG. 34, a superior and/or inferior plate member 650 for use in surgical implant 600 is shown. Plate member 650 includes a pair of slidable members 652, 654 and an aperture 656a formed in a center member 656 for accepting an insert component (not shown) of another plate member. The pair of slidable members 652, 654 may be configured to slide linearly and/or curvilinearly. The sliding paths of each of the pair of slidable members 652, 654 are configured to be substantially perpendicular to each other. Aperture 656a of center member 656 may allow one of the superior/inferior plate members to rotate relative to plate member 650.

Turning now to FIGS. 35 and 36, a surgical implant and/or plating system according to another embodiment of the present disclosure, for selective or permanent fixation to a pair of adjacent vertebra 10, is generally designated as 700. Surgical implant 700 includes a first or superior plate member 710, and a second or inferior plate member 720 in juxtaposed relation to superior plate member 710 and operatively associated therewith.

As seen in FIGS. 35 and 36, superior plate member 710 includes a plate-like body 712 which may have a substantially arcuate or concave profile. Body 712 defines at least one aperture 714 extending therethrough for receiving fastening members (not shown) used to fix or secure superior plate member 710 to a vertebra 10 (see FIGS. 1 and 2). Body 712 defines a longitudinal plane and defines a recess or passage 713a formed in a side surface thereof. Passage 713a may define a plurality of bearing surfaces including, and not limited to, a rear bearing surface 713b, a front bearing surface 713c, and side bearing surfaces 713d.

Inferior plate member 720 includes a plate-like body 722 having a substantially arcuate or concave profile. Body 722 defines at least one aperture 724 extending therethrough for receiving fastening members (not shown) used to fix or secure inferior plate member 720 to a vertebra 10 (see FIGS. 1 and 2) adjacent to the vertebra to which superior plate member 710 is fixed. Body 722 defines a longitudinal plane and includes a tongue or stem portion 722a extending substantially in the direction of the longitudinal plane, in juxtaposed relation to passage 713a formed in superior plate member 710. A distal end of stem portion 722a is configured to be operatively received in passage 713a of superior plate member 710. Stem portion 722a may have a circular or rectangular transverse cross-sectional profile.

In use, when surgical implant 700 is secured or fixed in the body, the distal end of stem portion 722a may bear against any one of rear bearing surface 713b, front bearing surface 713c, and side bearing surfaces 713d of passage 713a of superior plate member 710.

It is contemplated that stem portion 722a of inferior plate member 720 may be coated with a layer of cobalt chrome and/or the bearing surfaces of passage 713a of superior plate member 710 may be lined or coated with a layer of cobalt chrome.

It is further contemplated that passage 713a may be open ended in order to facilitate the manufacturing process, however, it is envisioned that passage 713a may be fabricated as a pocket, cavity, hollow or the like.

Stem portion 722a may be provided with a stop member 722b (see FIG. 36) disposed proximate a distal end thereof to prevent superior plate member 710 from separating from inferior plate member 720.

Surgical implant 700 is configured to allow for a minimum distraction (as a result of the bearing surfaces between tongue 712a of superior plate member 710 and body 722 of inferior plate member 720), as well as axial rotation and lateral bending (due to the configuration and dimensions of stem portion 722a and passage 713a).

It is contemplated that surgical implant 700 may be supplied fully assembled or, alternately, surgical implant 700 may be assembled in the operating room prior to or during the surgical procedure. Surgical implant 700 may be configured such that once surgical implant 700 is assembled, disassembly could be prohibited. It is further envisioned that a special tool may be required to separate or disassemble plate members 710, 720 and stem portion 722a from one another. It is contemplated that stem portion 722a be suitably fixed to superior plate member 710 and/or inferior plate member 720 in such a manner so as to withstand the fatigue life thereof.

The surgical implant could also have two parallel bearing surfaces, see FIG. 28, this could be done to move the load bearing components of the implant off of the centerline. This would allow a thinner design at the center of the plate were the plate will rub the esophagus when implanted. The thinner design could reduce post operative complaints of dysphasia.

Turning now to FIGS. 37-39, a surgical implant, for selective or permanent fixation to a pair of adjacent vertebra 10, is generally designated as 800. Surgical implant 800 includes a first or superior plate member 810, and a second or inferior plate member 820 in juxtaposed relation to superior plate member 810 and operatively associated therewith.

As seen in FIGS. 37-39, superior plate member 810 includes a plate-like body 812 which may have a substantially arcuate or concave profile. Body 812 defines at least one aperture 814 extending therethrough for receiving fastening members (not shown) used to fix or secure superior plate member 810 to a vertebra 10 (see FIGS. 1 and 2).

Body 812 defines a longitudinal plane and includes a tongue portion 812a extending substantially in the direction of the longitudinal plane. Tongue portion 812a defines at least one cut-out or recess 812b formed in an edge surface thereof. As seen in FIG. 39, each recess 812b may be defined by a top wall, a bottom wall and a pair of side walls which may be parallel to one another. In an embodiment, a pair of recesses 812b is provided.

As seen in FIG. 39, superior plate member 810 may include at least one plug 816 extending or projecting form a rear surface thereof, i.e., a surface with is to come into contact with vertebra 10.

As seen in FIGS. 37 and 38, inferior plate member 820 includes a plate-like body 822 having a substantially arcuate or concave profile. Body 822 defines at least one aperture 824 extending therethrough for receiving fastening members (not shown) used to fix or secure inferior plate member 820 to a vertebra 10 (see FIGS. 1 and 2) adjacent to the vertebra to which superior plate member 810 is fixed. Body 822 defines a longitudinal plane and includes at least one tongue portion 822a extending substantially in the direction of the longitudinal plane, from a side edge thereof.

Each tongue portion 822a of inferior plate member 820 has a transverse cross-sectional profile which may substantially similar to or complementary of recesses 812b of superior plate member 810. Alternately, each tongue member 822a may have a transverse cross-sectional profile which is smaller than the configuration of recesses 812b of superior plate member 810. In this manner, superior plate member 810 and inferior plate member 820 are capable of lateral movement or rotation relative to one another. In an embodiment, a pair of tongue portions 822a is provided, wherein each tongue portion 822a is configured and dimensioned for slidable receipt in a respective recess 812b of superior plate member 810.

As seen in FIG. 39, superior plate member 810 may include a friction increasing feature 818 disposed within and/or projecting into recesses 812b. In use, friction increasing features 818 will contact or engage an outer surface of tongue portions 822a of inferior plate member 820.

When superior plate member 810 and inferior plate member 820 are coupled to one another, superior plate member 810 and inferior plate member 820 are capable of translating relative to one another.

A method of performing a surgical procedure, with any of the afore-described surgical implants, includes the steps of accessing the site of the spine to be treated; fixing one side of a surgical implant to one vertebra; and fixing the other side of the surgical implant to another vertebra. The vertebrae can be distracted prior to placement of the surgical implant, and, when placed, the surgical implant would maintain the distraction. The surgical procedure is unique in that no preparation of the disc space, placement of bone graft, or artificial graft material is required. The surgical implant can be placed with or without removal of any osteophytes on the vertebra. In one method, no bone removal is required.

The method of fixation of the surgical implant is important in a dynamic construct as the implant system must function for long periods of time as designed, ideally over 10 years. It is desirable that the fixation means are designed not to loosen over time. The standard fixation means is a screw 30 (see FIG. 22). While a screw 30 may be a preferred embodiment of fixation in accordance with the present disclosure, such screws 30 must be modified to provide better long term fixation. One method is to manufacture screws 30 of titanium with a surface thereof roughened by bead blasting or other common techniques. Fixation may also be improved by coating screws 30 with hydroxy apatite.

The fixation means may have an insertion shape, usually a cylinder or screw, that can be inserted easily into bone by a pining or screwing action. The fixation means may have a second shape that more rigidly secures the surgical implant to the bone.

Some fixation means or devices have already been described in patents, for instance, a cannulated screw having a split at an end thereof, wherein at the end of the split the cannulation narrows. A rod may be inserted inside the cannulation and advanced past the point where the cannulation narrows. If the rod is greater in diameter than the narrowed cannula, the rod forces the split sections of the screw outward. This “splay” increases the fixation in bone.

Other fixation means could be mechanical or they could be manufactured from a shape memory material which when the material was implanted and its temperature reaches body temperature, the fixation means would move from the insertion shape to the fixed shape.

One method envisioned is similar to a drywall anchor. Still another is to insert a plate inside the bone which will lock with the screw. Finally another potential method has flanges that flare out after insertion.

If the screws are coated with hydroxy apatite or simply roughened a retention mechanism may be required to prevent the screws or other fixation means from backing out of the plate. Many methods can be used, any of which would likely be satisfactory for the present disclosure. These methods include a second plate to cover the screw heads or a slideable plate or bar which the surgeon can slide over to obstruct the fixation means from backing out. Another method uses a ring cut in the fixation means and a retention ring in the plate such that when the fixation means is advanced into the plate the retention ring locks into the cut on the fixation means.

One or more screws could be used on each member of the surgical implant, preferably two or three screws will be used on each member of the surgical implant. The screws could allow for variability in the insertion angle, but must be locked at a fixed angle in the final implanted state. Because fixation is a concern, the preferred embodiment has fixed angle screws that are locked relative to the plate member in the final configuration.

All of the surgical implants disclosed herein may be machined from biocompatible metals and/or polymers. The polymers can be molded and machined to the final shape desired or even molded to the final shape. Where different materials are used to manufacture a single part, said parts may be dovetailed, pinned or screwed together. Parts with cavities can also be manufactured out of two pieces then pinned, screwed or otherwise fixed together. If the parts are from similar compatible material they could also be welded together.

While the surgical implants described hereinabove have been described with reference to superior and inferior plate members for fixation to respective superior and inferior vertebrae, it is contemplated and understood that, depending on the physicians desire and/or the anatomy of the patient, that the superior and inferior plate members may be inverted such that the superior plate member is fixed to an inferior vertebrae and that the inferior plate member is fixed to a superior vertebrae.

The above described embodiments of the present invention are merely descriptive of its principles and are not to be limiting. The scope of the present invention instead shall be determined from the scope of the following claims, including their equivalents.

What is claimed is:

1. A spinal plating system for facilitating stabilization of the spine, the spinal plating system comprising:
superior and inferior plate members for positioning against exterior surfaces of respective superior and inferior vertebrae and in contacting relation therewith, said superior and inferior plate members including cooperating contacting surfaces adapted to engage each other to maintain a minimum predetermined distraction distance between the superior and inferior vertebrae and for permitting at least one of relative articulating movement, pivoting movement or lateral movement of the superior and inferior plate members; and
at least one fastener for mounting each of the superior and inferior plate members to the respective superior and inferior vertebrae.

2. A spinal plating system according to claim 1, wherein the superior and inferior plate members include corresponding stop surfaces to limit the at least one of relative articulating movement, pivoting movement or lateral movement of the superior and inferior plate members.

3. A spinal plating system according to claim 1, wherein one of the superior and inferior plate members includes a stop depending therefrom and the other of the superior and inferior plate members includes inner surface portions defining a recess for receiving the stop, whereby the stop is engageable with the inner surface portions to limit the at least one of relative articulating movement, pivoting movement or lateral movement of the superior and inferior plate members.

4. A spinal plating system according to claim 1, wherein the contacting surface is generally arcuate.

5. A spinal plating system according to claim 1, wherein positioning against exterior surfaces of respective superior and inferior vertebrae is accomplished at an anterior surface, a posterior surface, a lateral anterior surface or a lateral posterior surface of said vertebrae.

6. A spinal plating system according to claim 1, wherein each of the superior and inferior plate members are formed in a plurality of pieces which are configured for selective mating with one another.

7. A spinal plating system according to claim 1, wherein at least one of the superior plate member and the inferior plate member includes a pin projecting from a surface thereof and configured for operative engagement with a complementary feature formed on the other of the superior plate member and the inferior plate member.

8. A spinal plating system according to claim 7, wherein the complementary feature is a window having a dimension, and wherein the movement of the pin within the window is defined by the dimensions of the window.

9. A spinal plating system according to claim 1, wherein one of the superior and the inferior plate member includes a hollow formed in a surface thereof, and the other of the superior and the inferior plate member includes a stem having an enlarged head portion configured for receipt within the hollow.

10. A spinal plating system according to claim 9, wherein the hollow includes a rim formed at least partially around an end thereof for inhibiting removal of the head portion from within the hollow.

11. A spinal plating system according to claim 9, wherein the head portion is separable from the respective superior and inferior plate member.

12. A spinal plating system according to claim 1, further comprising an inter-engagement member operatively connecting the superior plate member and the inferior plate member to one another, wherein the inter-engagement member is rotatably connected to the superior plate member, and at least one of rotatably and slidably connected to the inferior plate member.

13. A spinal plating system according to claim 12, wherein the inferior plate member defines a cut-out and includes a bar extending across the cut-out, wherein the inter-engagement member is rotatably and slidably supported on the bar of the inferior plate member.

14. A spinal plating system according to claim 1, wherein one of the superior and the inferior plate members includes at least one recess formed in a side edge thereof, and the other of the superior and inferior plate members includes at least one respective tongue portion configured and dimensioned for slidable engagement in a respective recess.

15. A spinal plating system according to claim 14, wherein a pair of spaced apart recesses are provided, and a pair of complementary spaced apart tongues are provided.

16. A spinal plating system according to claim 14, wherein the recesses and the tongues have a substantially arcuate longitudinal axis.

17. A spinal plating system according to claim 16, wherein the tongues are substantially parallel to one another.

18. A spinal plating system according to claim 14, wherein at least one of the superior and the inferior plate member includes at least one plug projecting from a surface thereof for engagement with a vertebrae.

19. A spinal plating system for facilitating stabilization of adjacent vertebrae, the spinal plating system comprising:
superior and inferior plate members for mounting against respective superior and inferior vertebrae of a spine in contacting relation therewith, the superior and inferior plate members including cooperating contacting surfaces adapted to engage each other to maintain a minimum predetermined distraction distance between the superior and inferior vertebrae and for permitting relative movement of the superior and inferior plate members along at least two of an x-axis, y-axis and z-axis of the superior and inferior plate members; and
at least one fastener for mounting each of the superior and inferior plate members to the respective superior and inferior vertebrae.

20. A spinal plating system for facilitating stabilization of adjacent vertebrae, the spinal plating system comprising:
superior and inferior plate members for mounting against respective superior and inferior vertebrae of a spine in contacting relation therewith, the superior and inferior plate members joined to one another by an intermediate portion for maintaining a minimum predetermined distraction distance between the superior and inferior vertebrae and for permitting relative movement of the superior and inferior plate members along at least one of an x-axis, y-axis and z-axis of the superior and inferior plate members; and
at least one fastener for mounting each of the superior and inferior plate members to the respective superior and inferior vertebrae.

21. A spinal plating system according to claim 20, wherein the intermediate portion is formed of a resilient material.

22. A spinal plating system according to claim 21, wherein at least one of the superior and inferior plate member includes a stop member configured to prevent movement of the superior plate member and the inferior plate member relative to one another after the superior plate member and the inferior plate member have move a predetermined distance relative to one another.

23. A spinal plating system according to claim 21, wherein the intermediate portion includes at least one of at least one side cut formed in side surfaces thereof and at least one passage extending therethrough.

24. A spinal plating system according to claim 20, wherein the intermediate portion is formed in one of the superior plate member and the inferior plate member and is made of at least one of nitinol, an elastic material and a flexible material including at least one passage formed therein.
